SEQUENCE LISTING (2 sequences)

SEQ ID NO: 1 - DNA, 85 bp, Artificial
  5′ UTR region added by two step PCR method
gatcttaaggctagagtactaatacgactcactatagggaatacaagctacttgttctttttgcactcgagaattcgccaccatg

SEQ ID NO: 2 - DNA, 100 bp, Artificial
  3′ UTR region added by two step PCR method
taataacgactcaggctgctacaaaaaaaaaaaaaaaaaaaaaaaaaaaaaactagcataaccccttggggcctctaaacgggtcttgaggggttttttg

(19) United States
(12) Patent Application Publication
(10) Pub. No.: US 2007/0248971 A1
(43) Pub. Date: Oct. 25, 2007

(54) Programming microfluidic devices with molecular information

(75) Inventors: Sebastian J. Maerkl, Palo Alto, CA (US); Stephen R. Quake, Stanford, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: California Institute of Technology, Pasadena, CA (US)

(21) Appl. No.: 11/698,802

(22) Filed: Jan. 26, 2007

Related U.S. Application Data
(60) Provisional application No. 60/762,344, filed on Jan. 26, 2006.
(60) Provisional application No. 60/762,330, filed on Jan. 26, 2006.

(51) Int. Cl. G01N 33/00; B01L 11/00; B29C 65/48; C12Q 1/68

(52) U.S. Cl. 435/6; 156/60; 422/99; 435/41; 436/86

(57) ABSTRACT

The invention provides a microfluidic device having a plurality of chambers each containing separately deposited reagents. The invention also provides an efficient PCR-based method for producing a linear expression template. The invention also provides methods for analyzing interactions between molecules, involving flow-deposition of expression templates on the substrate of chambers in a microfluidic device, and expressing proteins from the templates.

### CROSS-REFERENCE TO RELATED APPLICATIONS

This application claims priority to U.S. Provisional Application No. 60/762,330 entitled “Mechanically Induced Trapping of Molecular Interactions” and Provisional Application No. 60/762,344 entitled “Programming Microfluidic Devices with Molecular Information,” both filed Jan. 26, 2006, and to U.S. Provisional Application No. 60/______ entitled “Mechanically Induced Trapping of Molecular Interactions” (Attorney Docket No. 20174C-016210 and to U.S. Provisional Application No. 60/______ entitled “Programming Microfluidic Devices with Molecular Information” (Attorney Docket No. 20174C-016310), both filed Jan. 11, 2007. The entire content of each of these applications is incorporated herein by reference.

### STATEMENT REGARDING FEDERALLY SPONSORED RESEARCH

Work described herein has been supported, in part, by the Office of Naval Research (ONR)—Space and Naval Warfare Systems Center (Grant No. N66001-02-1-8929; Subcontract Princeton 341-6260-515). The United States Government may have certain rights in the invention.

### FIELD OF THE INVENTION

The invention relates to novel microfluidic devices and methods of using them. The invention finds application in the fields of biology, chemistry, medicine and microfluidics.

### BACKGROUND

The use of microfluidic devices provides many advantages over classical benchtop methods, including an unrivaled economy of scale, new fluid dynamics and physics, as well as a high degree of parallelization and integration. All of these characteristics are based on the fact that microfluidic devices shunt liquids in channels with widths on the order of tens to hundreds of microns. Decreasing the size of fluidic devices not only has beneficial effects as stated above, but also creates problems due to the discrepancies in length scales between the device and the rest of the lab, including the researcher, complicating addressing devices and introduction of information in the form of reagents. Some of these issues have been solved including on-chip addressing of large numbers of flow channels as well as mixing of fluids in a pair wise fashion to generate a matrix of different reactions (see Thorsen et al., 2002, “Microfluidic large-scale integration” Science 298:580-4; Liu et al., 2003, “Solving the world-to-chip interface problem with a microfluidic matrix” Anal Chem 75(18):4718-23; and published US patent application US2004112442). The introduction and sequential mixing of a semi-large number, in the range of 10 to 100, of liquids has been demonstrated as well (Hansen et al., 2002 “A robust and scalable microfluidic metering method that allows protein crystal growth by free interface diffusion. Proc Natl Acad Sci USA, 99(26):16531-6 and citations supra). Introduction of larger numbers of compounds—on the order of hundreds or thousands—has been prohibitively problematic, and specific and defined introduction of a large number of compounds and the combinatoric downstream processing has not been accomplished to date.

### BRIEF SUMMARY OF THE INVENTION

In one aspect, the invention provides a method of fabricating a microfluidic device comprising i) positioning (a) an elastomeric block comprising a plurality of chamber recesses and (b) a solid support comprising a microarray of discrete reagent-containing regions, so as to align each reagent-containing region with a chamber recess, ii) adhering the block to the upper surface of the solid support so as to produce a plurality of chambers, wherein in each chamber the upper surface of the solid support provides one surface of the chamber and the inner surfaces of a chamber recess provides other surfaces of the chamber; wherein each reagent-containing region contains two or more discrete subregions, and wherein at least two subregions in each reagent-containing region contain different reagents.

In one embodiment the the solid support is epoxy-functionalized glass. In one embodiment each discrete reagent-containing region contains three discrete subregions, each of which contains a different reagent. In one embodiment each discrete reagent-containing region contains four or more discrete subregions.

In one embodiment, the reagents are deposited by contact printing. In one embodiment the microarray has a density of 100 or more discrete regions per cm². In one embodiment the microarray has a density of 1000 or more discrete regions per cm² In one embodiment microarray includes 10 to 500 different reagents. In one embodiment the reagents are proteins and/or nucleic acids.

In one aspect the invention provides a microfluidic device comprising a plurality of isolated reaction sites, wherein one surface of the reaction site is formed by a solid support and each isolated reaction site comprises a reagent-containing region on said surface, wherein the reagent-containing regions contain two or more discrete subregions, and wherein at least two subregions in each reagent containing region contain different reagents. In one embodiment the device comprises a plurality of reaction chambers wherein one surface of chamber is formed by a solid support and said surface comprises a reagent-containing region wherein the reagent-containing region contains two or more discrete subregions, and wherein at least two subregions in each reagent containing region contain different reagents. In one embodiment the solid support is epoxy-functionalized glass.

In one aspect the invention provides a microfluidic device comprising a plurality of isolated reaction sites wherein one surface of the reaction site is formed by a solid support and each isolated reaction site comprises a reagent-containing region on said surface wherein the reagent-containing regions contain a first reagent deposited on the solid support and a second reagent deposited on top of the first reagent. In one embodiment the first and second reagents are different. In one embodiment the reaction sites on the array comprise a dilution series of one reagent.

In one aspect, the invention provides a microfluidic device, comprising (a) a first plurality of microfluidic flow channels each channel comprising a substrate; (b) a second plurality of microfluidic flow channels, each channel comprising a substrate, the second flow channels intersecting the first flow channels to define an array of reaction sites; wherein expression templates encoding proteins are immobilized on said substrates; and wherein at least one channel in the first plurality comprises an immobilized expression template that differs from the expression template immobilized in at least one channel in the second plurality; and (c) sets of isolation valves selectively actuatable to fluidically isolate reaction sites from each other, wherein said sets of valves each isolate a reaction region comprising a defined combination of expression templates, wherein the defined combinations each comprise an expression template that is immobilized in a channel from the first plurality and a different expression template that is immobilized in a channel from the first plurality.

In one embodiment, the isolated reaction regions comprise, in aggregate, at least 50 different defined combinations of expression templates. In one embodiment the number of unique expression templates in the first plurality of microfluidic flow channels is at least 10. In one embodiment the number of unique expression templates in the second plurality of microfluidic flow channels is at least 10. In one embodiment the number of unique expression templates in the first plurality and second plurality of microfluidic flow channels, taken together, is at least 10. In one embodiment the device additionally comprises at least one set of intersecting channels in which both channels in the set comprise the same expression template.

In one aspect the invention provides a method for analyzing protein-protein interactions comprising (i) in a microfluidic device as described above introducing a cell-free transcription translation system into the regions comprising defined combinations of expression templates, actuating valves to isolate reaction regions, and maintaining the device under conditions in which protein synthesis occurs and thereby producing proteins encoded by the expression templates; and (ii) detecting the interaction between said proteins.

In one aspect the invention provides a method for producing a protein comprising, in a microfluidic device comprising a microfluidic flow channel comprising a substrate on which an expression template encoding a protein is immobilized; flowing a composition containing reagents sufficient for cell-free transcription and translation (ITT composition) through the channel, under conditions in which transcription of the expression template occurs and the encoded protein is produced; and, collecting the encoded protein from the flow channel. In one embodiment the ITT composition is Wheat Germ extract. In one embodiment the microfluidic device comprises a plurality of flow channels and wherein the expression templates immobilized in at least two of the flow channels is different.

### BRIEF DESCRIPTION OF THE FIGURES

FIG. 1 shows three approaches to printing micro-arrays for use with microfluidic devices. Spots indicate actual array spots and the label of the spot indicates the contents of the spot. Arrows if given indicate the preferred direction in which spots are deposited. Panel A depicts a standard micro-array where each spot is unique and originates from a unique solution. Panel B shows a co-multispotted pattern in which over three rounds a three dimensional matrix is generated. First columns are spotted with the solutions A and B respectively, followed by spotting of solutions 1 and 2 in the respective rows directly on top of the previously spotted solutions. In the third round solutions alpha and beta are spotted. Panel C shows an array similar to that shown in Panel B, except that the spots are placed adjacent to, rather than on top of, one another.

FIG. 2 shows examples of arrays as described in their respective panels in FIG. 1. FIG. 2A shows a standard 2304 spot array of dsDNA oligomers labeled with Cy5. FIG. 2B shows a 200 spot co-spotted array of various Cy3 labeled linear expression templates shown on the left and a dilution series of dsDNA oligomers labeled shown on the right. Both images were taken of the same array at two different wavelengths, with Cy3 on the left and Cy5 on the right. FIG. 2C shows a neighbor-array with 320 unit spots and 640 total spots. Each unit spot consists of two different linear expression templates labeled 9 with Cy3.

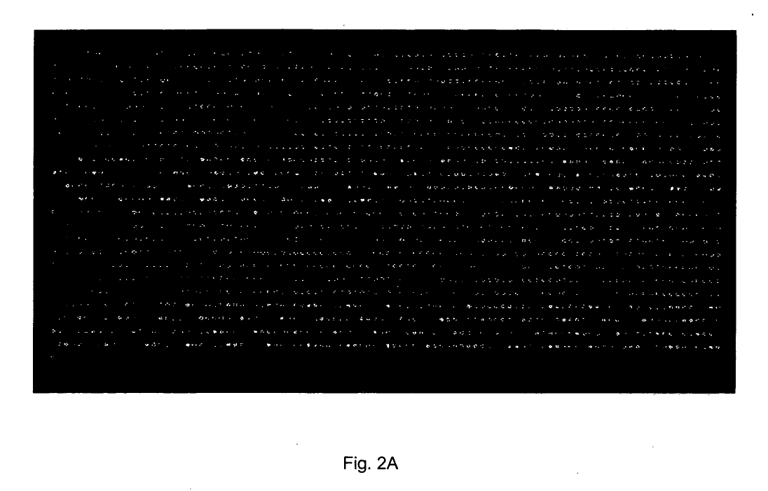

FIG. 3 shows a microfluidic device (DTPAx8). FIG. 3A: AutoCad schematic of a microfluidic device (DTPAx8). The device consists of two fluidic layers, the control layer (red) situated positioned on top of the flow layer (blue). The control is used to address valves which shunt liquid on the flow layer. FIG. 3B shows an actual device with control lines filled with food dyes of various color. The flow lines are empty and thus transparent.

FIG. 4 shows a blow-up of one of the 640 unit cells taken from the same device as in FIG. 3B. The active valves and free-standing membrane are numbered. Valves #1 are used for the segregation of the individual unit cells, valve #3 protects the chamber from filling with fluid while other steps are being performed and the free-standing membrane #2 is used for surface derivatization and MITOMI.

FIG. 5 shows a false color fluorescent scan of a DNA microarray aligned to a microfluidic device similar to the one shown in FIG. 3 with slightly varied unit cell. When reproduced in color, yellow to orange DNA spots are clearly visible and are all aligned to a circular microfluidic chamber.

FIG. 6 shows three schemas that use flow deposition to generate complex combinatoric assay on a microfluidic device. FIG. 6A shows the simplest and not truly combinatoric approach which serves as the principal component on which all other methods are based. Here three linear expression templates A through B are deposited in three parallel flow channels. This approach may be combined with the spotting based programming approach described above to give rise to a combinatoric array shown in FIG. 6B. If a second set of perpendicular flow channels is derivatized with a second set of linear templates a matrix is also generated as shown in FIG. 6C.

FIG. 7: FIG. 7A is an AutoCad design of the Binary Interaction Chip v2 (BICv2). The flow layer (blue) and control layer (red) are identified. FIG. 7B shows a fluorescent scan of the device with flow deposited linear expression template DNA. Each column and row contains the indicated linear template. Here the intensity scales from white to black with black being the highest intensity.

FIG. 8: Two step PCR method for generating linear expression templates.

FIG. 9: 5′ and 3′ UTR sequences added by the two step PCR method. All regions are annotated and all priming sequences are underlined. The start and stop codons are double underlined. The entire 5′ and 3′ UTRs are added by the 5′extension and 3′extension primers respectively except for the start and stop codons.

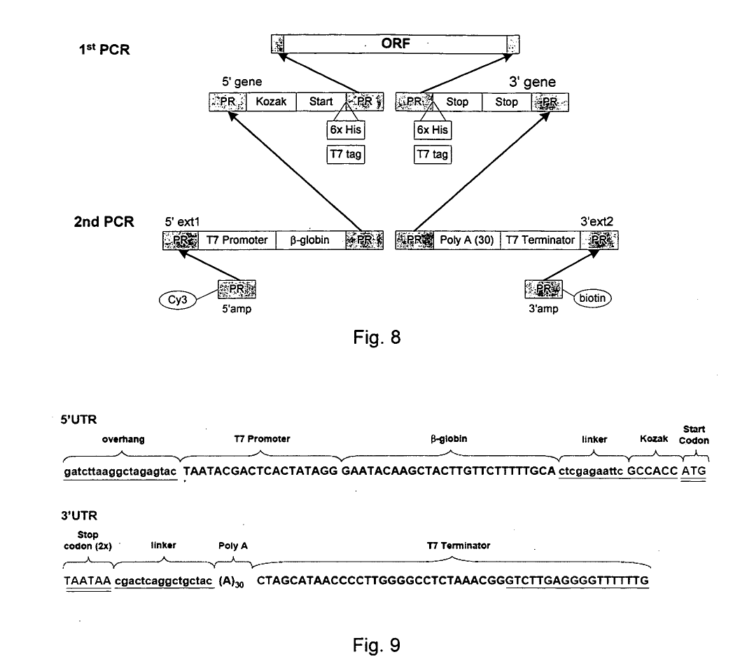

### BRIEF DESCRIPTION

In PART 1 we present methods for the programming of microfluidic devices with a large number of distinct reagents (e.g., thousands to tens of thousands or more per device). This method is based on generating spotted microarrays on a substrate, and aligning the substrate with a microfluidic device containing channels, valve, chambers pumps, etc. (i.e., “plumbing”) so that individual reagent-containing spots and combinations of spots are compartmentalized and segregated from the rest of the array.

In PART 2 we present a method for PCR-based amplification to produce a linear expression template. The method is highly modular and can easily be scaled up to thousands of target genes. Our approach only requires an open reading frame (ORF) as starting material, which may be obtained from a variety of sources including yeast and bacterial genomic DNA or eukaryotic cDNA clones.

In PART 3 we present a method for the flow dependent surface deposition of expression templates (e.g., linear expression templates) to be used in in vitro transcription/translation for the in situ generation of protein. Expression of the template encoded proteins may be used for highly efficient in vitro protein synthesis. Also presented are methods for deposition of multiple templates. Using this method it is possible to analyze a vast number of protein interactions. For example, all possible combinations of binary interactions between the two sets of proteins can be analyzed rapidly and inexpensively.

These methods find use in a variety of applications including, but not limited to, Mechanically Induced Trapping of Molecular Interactions (MITOMI) which is described in copending application No. 60/______ entitled “Mechanically Induced Trapping of Molecular Interactions” (Attorney Docket No. 20174C-016210, filed Jan. 11, 2007, and application Ser. No. 11/______ entitled “Mechanically Induced Trapping of Molecular Interactions” (Attorney Docket No. 20174C-016220), filed Jan. 26, 2007. The entire content of each of these applications is incorporated herein by reference. MITOMI and other methods related to the present disclosure are also described in Maerkl S J and Quake S R, 2007, “A systems approach to measuring the binding energy landscapes of transcription factors” Science 315:233-7, incorporated herein by reference.

The methods disclosed herein may be used using microfluidic devices. Materials and methods for producing a variety of microfluidic devices are known in the art. For illustration and not limitation, a brief discussion of useful methods is provided infra in Part 4 (entitled “General Materials and Fabrication Methods”). In some embodiments the methods are carried out using an elastomeric microfluidic device using MSL fabrication techniques (see, e.g., Unger et al., 2000, “Monolithic microfabricated valves and pumps by multilayer soft lithography” Science 288:113-16), although the methods are not limited to these specific devices. Elastomeric devices made using multilayer soft lithography (MSL) techniques are well known, and familiarity with such devices by the reader is assumed in the description herein.

### DEFINITIONS

As used herein, the term “microfluidic” device has its normal meaning in the art and refers to a device with structures (channels, channels, chambers, valves and the like) at least some of which have at least one dimension on the order of tens or hundreds of microns. In general, at least one structure of the device has dimension(s) below 1000 microns.

As used herein, “elastomeric” has its normal meaning in the microfluidic arts. Elastomers in general are polymers existing at a temperature between their glass transition temperature and liquefaction temperature. See Allcock et al., Contemporary Polymer Chemistry, 2nd Ed. Elastomeric materials exhibit elastic properties because the polymer chains readily undergo torsional motion to permit uncoiling of the backbone chains in response to a force, with the backbone chains recoiling to assume the prior shape in the absence of the force. In general, elastomers deform when force is applied, but then return to their original shape when the force is removed. The elasticity exhibited by elastomeric materials may be characterized by a Young's modulus. Elastomeric materials having a Young's modulus of between about 1 Pa-1 TPa, more preferably between about 10 Pa-100 GPa, more preferably between about 20 Pa-1 GPa, more preferably between about 50 Pa-10 MPa, and more preferably between about 100 Pa-1 MPa are useful in accordance with the present invention, although elastomeric materials having a Young's modulus outside of these ranges could also be utilized depending upon the needs of a particular application. Given the tremendous diversity of polymer chemistries, precursors, synthetic methods, reaction conditions, and potential additives, there are a huge number of possible elastomer systems that could be used to make the devices of the invention. Common elastomeric polymers include perfluoropolyethers, polyisoprene, polybutadiene, polychloroprene, polyisobutylene, poly(styrene-butadiene-styrene), polyurethanes, and silicones, for example, or poly(bis(fluoroalkoxy)phosphazene) (PNF, Eypel-F), poly(carborane-siloxanes) (Dexsil), poly(acrylonitrile-butadiene) (nitrile rubber), poly(I-butene), poly(chlorotrifluoroethylene-vinylidene fluoride) copolymers (Kel-F), poly(ethyl vinyl ether), poly(vinylidene fluoride), poly(vinylidene fluoride-hexafluoropropylene) copolymer (Viton), elastomeric compositions of polyvinyl chloride (PVC), polysulfone, polycarbonate, polymethylmethacrylate (PMMA), and polytertrafluoroethylene (Teflon), polydimethylsiloxane, polydimethylsiloxane copolymer, and aliphatic urethane diacrylate.

As used herein, the term “elastomeric block” refers to the elastomeric portion of a microfluidic device made using multilayer soft lithography techniques, which has not yet been adhered to a solid support (or substrate). The elastomeric block contains a plurality of recesses that, upon attachment of the solid support form chambers in which the solid substrate forms one surface (e.g., the “floor”). More generally, a “microfluidic block” is a partially fabricated microfluidic device having chamber recesses that form chambers or reaction areas when the block is adhered to the planar surface of a solid support on which an array is spotted. A “microfluidic block” does not necessarily comprise elastomeric components.

As used herein, the term “chamber recesses” of an elastomeric block refers to recesses that form chambers or reaction areas when the block is adhered to the planar surface of a solid support.

As used herein, “unit cell” refers to a combination of microfluidic structural elements that is repeated many times (e.g., 48 to 10,000 times, 100 to 5,000 times, or 250-2500 times) in a microfluidic device, where unit cells can operate simultaneously to carry out a function in a highly parallel manner.

As used herein, an “expression template” is a DNA molecule with a protein-encoding sequence (open reading frame) and operably linked sequences required for transcription and translation to produce the protein. These sequences, or elements, are known in the art and include a RNA polymerase start site for transcription, a ribosome binding site and associated regulatory structures, a start codon defining the start of the template, and optionally a stop codon, poly A tail, RNA polymerase stop sequence, sequences that extend the life of the mRNA (such as beta-globin sequence). The expression template may be linear or have a closed-circular topology (e.g., a template in a plasmid vector). Preferably the expression template is a linear double stranded molecule. In certain embodiments the expression template includes a covalently linked ligand (e.g., biotin) or other molecule that allows the template to be immobilized on a surface of a microfluidic channel. Ligands are easily introduced during synthesis of the template using PCR amplification methods. One suitable method is described hereinbelow in Part 2. Other methods are known in the art (e.g., Sawasaki et al., 2002, Proc. Natl. Acad. Sci. USA 99(23):14652; Lesley et al., 1991, J. Biol. Chem., 5: 2632., Watzele et al., 2001, Biochemica 3: 27-28; Lanar et al., 1996; Methods Mol. Biol. 66: 309-317; Burks et al., 1997; Proc. Natl. Acad. Sci. USA 2: 412-417, all incorporated herein by reference).

As used herein, “substrate” refers to a surface in a chamber or channel in a microfluidic device. Usually a chamber or channel can be defined by reference to substantially planar surfaces (e.g., floor, ceiling, and walls) and “substrate” refers to a particular planar surface, e.g., the “floor.” More particularly, “substrate” refers to an exposed surface and may change over time. For example, in a microfluidic chamber in which one surface is formed by a solid support (e.g., an epoxy-derivatized glass slide) coated with BSA, the substrate is the BSA layer.

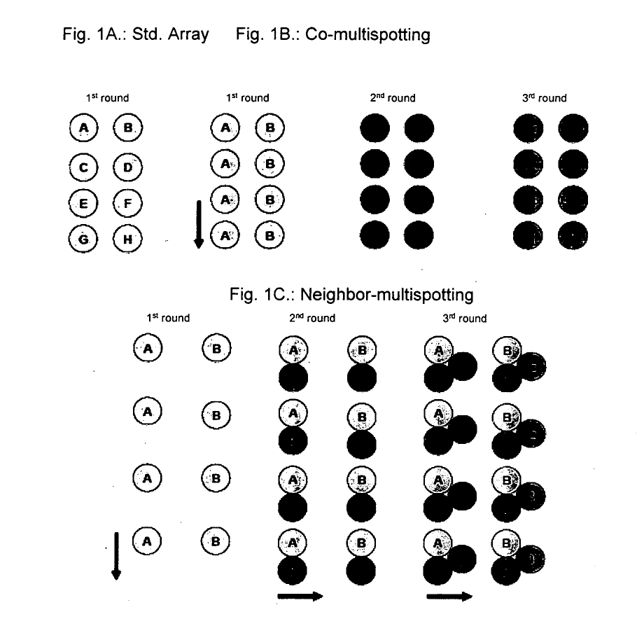

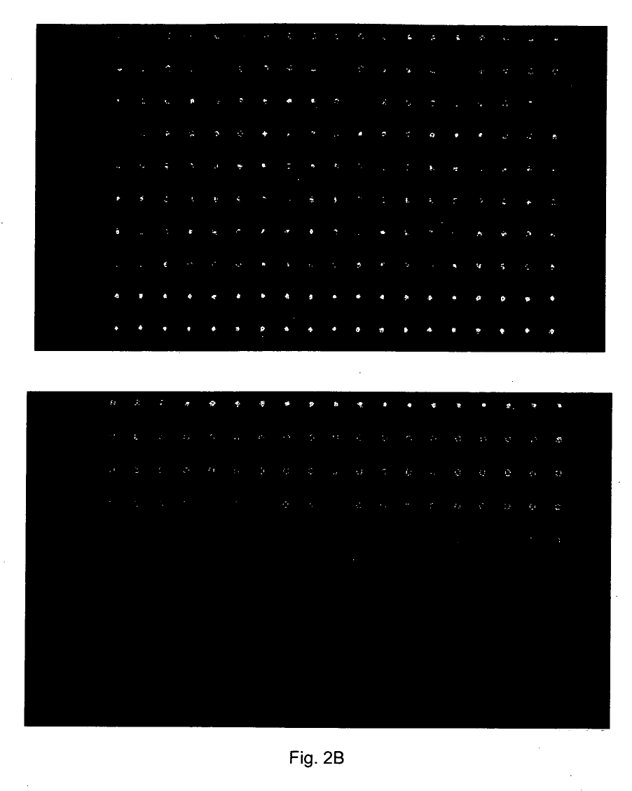

As used herein, the term “flow channel” refers to a microfluidic channel through which a solution can flow. The dimensions of flow channels can vary widely but typically include at least one cross-sectional dimension {e.g., height, width, or diameter) less than 1 mm, preferably less than 0.5 mm, and often less than 0.3 mm. Flow channels often have at least one cross-sectional dimension in the range of 0.05 to 1000 microns, more preferably 0.2 to 500 microns, and more preferably 10 to 250 microns. The channel may have any suitable cross-sectional shape that allows for fluid transport, for example, a square channel, a circular channel, a rounded channel, a rectangular channel, etc. In an exemplary aspect, flow channels are rectangular and have widths of about in the range of 0.05 to 1000 microns, more preferably 0.2 to 500 microns, and more preferably 10 to 250 microns. In an exemplary aspect, flow channels have depths of 0.01 to 1000 microns, more preferably 0.05 to 500 microns, more preferably 0.2 to 250 microns, and more preferably 1 to 100 microns. In an exemplary aspect, flow channels have width-to-depth ratios of about 0.1:1 to 100:1, more preferably 1:1 to 50:1, more preferably 2:1 to 20:1, and most preferably 3:1 to 15:1, and often about 10:1. A flow channel need not have a uniform width along its length and, as described below, may be wider in the region in which the detection area is situated in order to accommodate a trapping membrane or other trapping element. For example the portion of the channel substrate that contains the detection region may be widened (i.e., wider than other portions of the flow channel) and/or rounded (see FIG. 1A and FIG. 3).

### Part 1

### Programming Microfluidic Devices Using Multiply-Spotted Arrays

In PART 1 we present methods for the programming of microfluidic devices with a large number of distinct reagents (e.g., thousands to tens of thousands or more per device). This method is based on generating spotted microarrays on a substrate, and aligning the substrate with a microfluidic device containing channels, valve, chambers pumps, etc. (i.e., “plumbing”) so that individual reagent-containing spots or groups of spots are compartmentalized and segregated from the rest of the array (i.e., other spots or groups of spots in the array). Using this method, as many individual reactions can be run per chip as there are spots and chambers.

We also present a variation of this method in which more than one compound is tested in each chamber. This method involves spotting two or more different compounds (i.e., “double- or multiple-spotting”) either directly on top of or immediately adjacent to one another, such that each chamber contains two or more spots.

This approach allows for the facile generation of several thousand complex assays with multiple distinct species taking part in each reaction. A second important aspect of the invention is the fact that any soluble substance may be thus spotted and tested. Additionally any colloidal particle, such as quantum dots, bacterial cells, beads (e.g., silica beads) and viral particles may also be introduced into the devices. This provides a method for the high-throughput content screening of most any liquid based library that can be imagined, with the added benefit of microfluidics enabling the compound library to be tested using complex fluidic assays.

The methods of the invention can be carried out using any type of microfluidic device that contains channels in which one surface is a planer solid support on which an array can be spotted, and contains valves that allow regions of the channels to be fluidically isolated from other regions thereby forming chambers. In one embodiment elastomeric microfluidic devices fabricated by multilayer soft lithography (MSL) are used. These devices include a first elastomeric layer with microfabricated recesses having a width less than 1000 micrometers (the flow layer); a a second elastomeric layer with microfabricated recesses having a width less than 1000 micrometers (the control layer) bonded together to form a monolithic elastomeric block having a control layer containing control channels and a flow layer containing recesses. The elastomeric block is adhered to the upper surface of a solid support thereby creating flow channels in which one inner surface of each flow channel is the upper surface of the solid support, and other inner surfaces of each the flow channel the inner surfaces of the microfabricated recesses. A portion of the monolithic elastomeric block is deflectable into the flow channels.

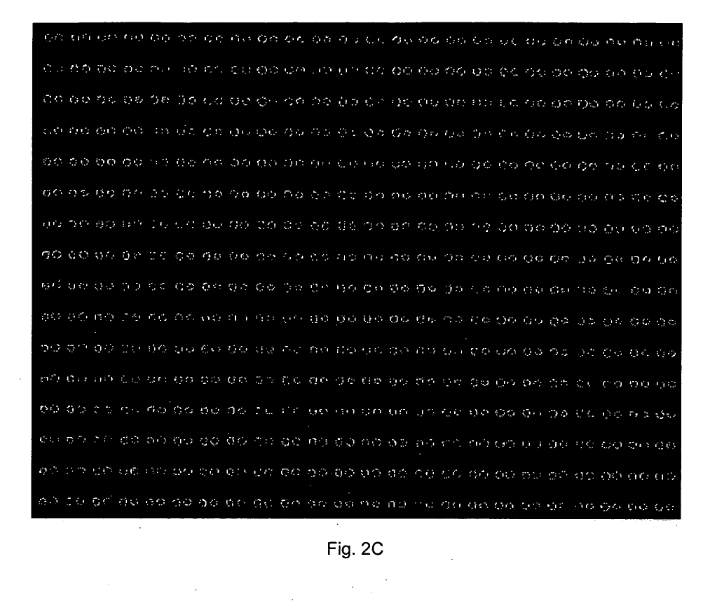

Reaction sites are sites at which a molecular interaction (binding, disassociation, formation or breaking of covalent bonds, change in state, etc.) occurs. Reaction sites can exist within a flow channel, at the intersection of two flow channels, or at the end of a dead-end channel, for example, when valves are closed to fluidically isolate the reaction site. An “isolated reaction site” generally refers to a reaction site that is not in fluid communication with other reactions sites present on the device (i.e., one or more valves are closed to isolate the site). A reaction site may exist within a “reaction chamber.” As used herein, the term “reaction chamber” refers to the terminal portion (the “dead end”) of a blind flow channel. A “blind channel” or a “dead-end channel” refers to a flow channel which has an entrance but not a separate exit. Accordingly, solution flow in and out of the blind channel occurs at the same location. Blind channels are described in U.S. Pat. No. 7,118,910, incorporated herein by reference for all purposes. In some embodiments the reaction chamber is located at the terminus of a flow channel but is broadened so that the chamber has greater surface area than a corresponding length of a flow channel. For example, a reaction chamber may have the configuration of the round chamber shown in FIG. 4, which can be isolated by closing valve 3. See, e.g., Unger et al., 2000, Science 288:113-16; Thorsen et al., 2002, Science 298:580-584; Linger et al., 2000, Science 288:113-16; Quake & Scherer, 2000, Science 290:1536-40; U.S. Pat. No. 6,960,437; U.S. Pat. Nos. 6,899,137; 6,767,706; 6,752,922; 6,408,878; and 6,645,432; U.S. Patent Application publication Nos. 2004/0115838, 20050072946; 20050000900; 20020127736; 20020109114; 20040115838; 20030138829; 20020164816; 20020127736; and 20020109114; PCT patent publications WO 2005/084191; WO05030822A2; and WO 01/01025.

For illustration and not limitation, reaction sites and chambers often have volumes of about 0.5-1 nL. Exemplary isolated reaction sites and reaction chambers may have a generally circular footprint and have dimensions including a diameter of about 10 to about 1000 microns, e.g., from about 200-300 microns, e.g., about 250 microns, and heights of from about 1 to about 200 microns, e.g., about 5 to about 20 microns, e.b., about 10 microns. Chambers having a non-circular shape may have similar volumes.

Standard Arrays

We use spotted micro-arrays generated using standard technology based on quill pens which pick up defined amounts of liquid by capillary action and physically deposit drops on a substrate. Arrays are generated by a robot, which guides the pen or pens between a solution plate, generally a multiwell plate of 96, 385 or 1024 wells containing the reagents to be spotted, and the substrate. Conventional micro-arrays contain spots that have been deposited on the substrate such that each spot contains the content of a single well from which the liquid was picked up. In order to reduce cross-contamination between spots the pen is washed and dried before it is dipped into a new solution well to pick up the next solution to be spotted. A schematic of a small example array is shown in panel A of FIG. 1. In this schematic, eight different solutions, “A” through “H”, have been deposited in a square array. In reality, arrays may contain tens to hundred thousands of unique spots.

In order to facilitate re-solvation of the reagents a carrier may be introduced to facilitate re-solvation of the reagents. For example reagents may be co-spotted with a 1%-2% BSA solution. The co-deposited BSA also aids in the visualization of the spots necessary for the manual alignment of the array to the microfluidic chambers. Other additives such as other proteins, NaCl or other salts, PEG and other larger organic molecules may also be used as carriers.

Using this method each spot can be segregated such that individual and specific reactions may be run according to the spot composition and combinatorics introduced by the microfluidic device. This allows the micro-arrays to be used as true combinatoric assays rather than testing an entire array against a single input making our approach more powerful than conventional methods. Furthermore since the spots contained on the array are aligned to individual chambers, and the contents of the spots are being re-solvated rather then staying attached to the substrate it is possible to introduce more than one reagent per spot and ultimately allow the reagents to mix on-chip. This has the additional advantage that the assays performed using this method can be based on most any method developed for solution based bench-top chemistry rather then being limited to solid phase chemistry. Thus combinatorics may easily be achieved off-chip, reducing the fluidic complexity required on-chip to achieve similarly complex assays, ultimately allowing for higher assay densities and thus throughput by reducing the fluidic complexity required on-chip.

Using micro-arrays also presents an efficient method for introducing material into a microfluidic device, since the delivery volume and the microfluidic assay volumes are roughly matched. Reported spot delivery volumes are on the order of 1 nL, quite comparable to the on-chip chamber volumes of ˜0.5 nL and reaction chamber volumes of slightly bigger than 1 nL. Increasing sample concentration by co-multispotting of the same solution on the same spot is also an advantage and useful when reagent concentrations are low, which is the case with precious biological samples. Using the co-spotting method it is possible to increase reagent concentration per spot to levels that are useful and allow methods to be performed that otherwise would be impossible due to reagent limitations.

Alignment and Bonding

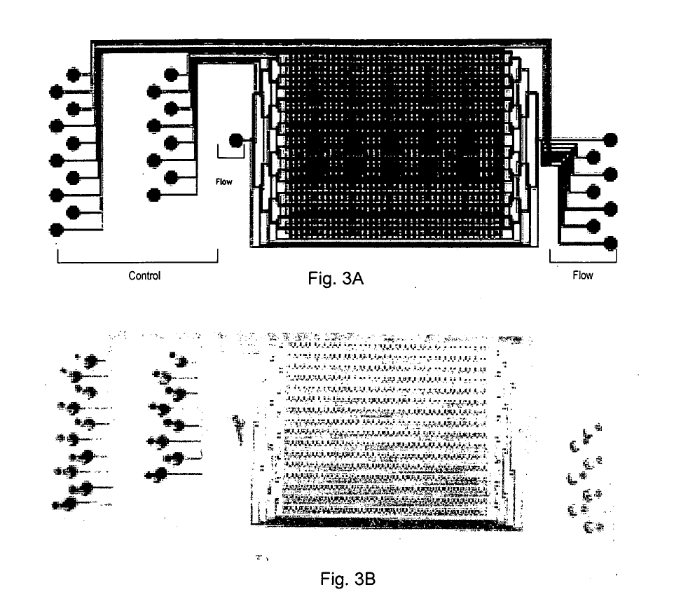

Once an array is spotted it is then aligned to a device such as the one depicted in FIG. 3, panel A. This device contains 640 dumbbell shaped unit cells shown in FIG. 4. A 640 spot micro-array can be aligned to the device such that each spot is located in one of the unit cell chambers (FIG. 3, Panel B bottom chamber). FIG. 5 shows an fluorescent scan of a device with a similar architecture aligned to a DNA microarray.

Bonding to the microfluidic PDMS device takes place via epoxy group present on the microarray glass slide. This reaction takes place at 40 degrees C. and also to a lesser degree at room temperature allowing temperature sensitive reagents such as DNA to be used in this process.

Re-Solvation

The desiccated reagent spots are thus introduced into the device and may be re-solvated by introducing liquid through the flow channel network. Once the reagents have gone into solution, valves (FIG. 4, valves #1) can be closed, segregating each unit cell on the device, avoiding cross-contamination between individual spots.

Complex Arrays

Since all the spots are ultimately segregated on the microfluidic device by our specific channel geometry and active valves it is possible to make efficient use of more complex arrays. Two approaches especially useful introduce more than one solution into the same vicinity, creating complex multiplexed arrays to be tested on-chip.

Co-Spotting

The first example is based on co-multispotting of the various solutions on top of one another in sequential rounds of spotting (Panel B FIG. 1). Here we generate all possible 3 solution combinations of 3 pairs of solutions, A or B and 1 or 2 and alpha or beta. The total number of possible combination is 8, or 2³. Specifically these arrays are generated by spotting duplicates of solutions in all the columns followed by a second round of spotting of the same or different solutions-into all the rows, also in duplicates. The first two rounds represent a standard two dimensional array of dimensions m×n where m is the number of columns and n the number of rows of the array. Printing of a three dimensional array of shape m×n×o can be accomplished by spotting as many copies of the 2 dimensional array m×n as the depth o of the 3 dimensional case. So in the case shown in panel B of FIG. 1 a 3-dimensional array of shape m=n=o=2 is spotted on a two dimensional substrate. Likewise any array of higher dimensionality can be printed using the same technique. The duplicates of solution A and B spotted in the same round may be spotted in sequence without the need of a wash step between duplicate spots, simplifying and thus speeding up the spotting. For any subsequent round of spotting it is desirable or necessary to wash between every deposited spot due to possible contamination of the pin from the previously deposited spot.

Co-multispotting is extremely space efficient since it requires the same area as a standard array, and spots may be spaced with a minimal pitch merely dictated by the pin, spotting robot and fluidic layout to which the array is being aligned.

Another interesting aspect of co-multispotting lies in the ability to increase reagent concentration per spot by multispotting the same solution several times on the same spot, each time delivering more reagent to the amount already present on the slide.

Neighbor Multispotting

A second approach to multiplexing by spot deposition is the method of neighbor-multispotting depicted in panel C of FIG. 1. Here instead of spotting the various solutions directly on top of one another, they are spotted immediately adjacent to one another. Each group of spots is then ultimately segregated on the device and the spotted solutions are allowed to mix by passive diffusion. This approach has the disadvantage of requiring a larger footprint per spot then the co-spotting method. Depending on the application this disadvantage might be far outweighed by the advantage that cross-contamination between spotting duplicates is eliminated. Eliminating cross-contamination in this fashion allows for drastic time savings in spotting larger arrays due to the reduction in required wash steps as well as allowing for the implementation of the co-multispotting of the same solution in order to increase reagent concentration as described above.

This method is broadly applicable to a wide variety of compounds, since the only prerequisite is that the compound to be spotted is soluble. Additionally it is beneficial if the solvent used is volatile to a certain extend, so that it evaporates and automatically dries the compound deposited. Likewise it should also be possible to align droplets to the microfluidic device. If drops are to be aligned they have to be compatible with the compound from which the microfluidic device is fabricated. Aside from any molecular compound, small colloidal particles such as quantum dots, bacterial cells and viral particles for example can also be deposited and used to program the device, making the spotting method extremely useful for a plethora of applications.

Using our approach for segregating microarrays has not been realized by other groups at this point, mainly because of the fact that microfluidic plumbing of considerable complexity is needed to achieve spot segregation. Furthermore aligning microarrays to microfluidic devices is not entirely trivial. Using spotted reagent arrays are generically used in solid phase detection, where the spotted reagents are affixed to the surface. In the approach described herein, the reagents are allowed to go into solution.

Co-spotting can be used increasing reagent concentration and multispotting of more than one reagent on a single spot.

In some embodiments of the invention, reagents are introduced onto a unit cell chamber by “spotting.” By using array technology, a different reagent or different combinations of reagents can be added to each unit cell. For example, a DNA array can be used in which each unit cell contains a different DNA sequence. This process involves: (1) obtaining (a) a solid support (e.g., an epoxy-coated glass slide), and (b) a microfluidic block (2) spotting one or more reagents on the solid support in a microarray pattern thereby producing a microarray of the reagents on the solid support; and (3) aligning the microarray to the partially fabricated microfluidic device and adhering the two to produce a microfluidic device having a substrate formed from the solid support and oriented so that each spot (or predetermined group of spots) of the array is located in a unit cell chamber of the device. In some embodiments reagents are deposited by contact printing; in other embodiments reagents are deposited by non-contact printing. The array usually has a density of at least 100 or more discrete regions per cm²; and sometimes has a density of at least 1000 or more discrete regions per cm². The array usually has a density of from 100 to 5,000 discrete regions per cm², most usually from 100 to 2000 discrete regions, often from 500 to 1500 discrete regions.

In one aspect, the invention provides a method of fabricating a microfluidic device by i) positioning an elastomeric block comprising a plurality of chamber recesses and a solid support comprising a microarray of discrete reagent-containing regions so as to align each reagent-containing region with a recess; ii) adhering the block to the solid support so as to produce a plurality of chambers containing reagents, where each reagent-containing region contains two or more discrete subregions, each containing a different reagent. The solid support may be a generally planer substrate made from any of a variety of materials such as, for example, an elastomer, glass (e.g., epoxy-functionalized glass), quartz, mica, or other materials. In one embodiment the support is transparent.

In one embodiment the solid support is an epoxy-functionalized glass slide, the partially fabricated microfluidic device is an elastomeric device formed from PDMS, and bonding occurs due to an attack of the electrophilic carbon of the epoxyde functional group by unreacted hydroxyl, alkoxyl or carboxyl groups of the PDMS. Bonding can be accelerated by heating the device to 40° C., or can be allowed to occur at room temperature. Other substrates include, for example and not for limitation, a tertiary layer of PDMS, unmodified glass, aldehyde surfaces (e.g., silylated slides from TeleChem International), plasma treated surfaces, etc.

Glass slides can be epoxy functionalized using 3-glycidoxypropyltrimethoxy silane, glycidoxypropyldimethoxymethylysilane, 3-glycidoxypropyldimethyl thoxyysilane or similar molecules (e.g., having an epoxy functional group linked to a silane group). In essence a silane molecule carrying a epoxyde functional group is either vapor deposited or absorbed in a liquid bath onto the glass surface where it the silane moiety covalently bonds to the glass surface. Vapor deposition simply involves vaporizing the above mentioned molecule (generally at room temperature as it is a volatile) in a small chamber to which the glass slides are added. In the liquid-dip process a roughly 1% solution of the above molecule in an organic solvent or mixture of organic solvent and water is used in which the slides are dipped until the surface has been coated with the above mentioned molecule. Epoxy coated slides are commercially available, e.g., CEL Associates (worldwideweb.cel-1.com), Telechem International (worldwideweb.arrayit.com), Xenopore Corp. (worldwideweb.xenopore.com).

The reagents deposited in the array can be any of a wide variety of compounds. In various embodiments the compound is selected from the following: DNA, RNA, proteins, peptides, antibodies, glycans, proteoglycans, receptors, cells, small organic molecules. Compounds that may be spotted include any soluble substance, or any suspension [e.g., cells (e.g., bacterial cells), or small particles such as quantum dots, beads (e.g., silica beads) and viral particles] that can be picked up and deposited by the arraying method used. The substrate in the area of deposition may be derivatized to bind or otherwise interact with the spotted reagent.

A wide variety of methods are known for producing arrays. See, for example, Heller, 2002, “DNA Microarray Technology: Devices, Systems, and Applications” Ann Rev Biomed Eng 4:129-53; Wingren & Borrebaeck, 2006, “Antibody microarrays: current status and key technological advances” OMICS 10:411-27; Oh et al., 2006, “Surface modification for DNA and protein microarrays” OMICS 10:327-43; Uttamchandani et al., 2006, “Protein and small molecule microarrays: powerful tools for high-throughput proteomics” Mol Biosyst. 2:58-68; and Uttamchandani et al., 2005, “Small molecule microarrays: recent advances and applications” Curr Opin Chem Biol. 9:4-13, each of which is incorporated herein by reference.

Technologies for forming microarrays include both contact and non-contact printing technologies. One example is the PixSys 5500 motion control system from Cartesian Technologies (Irvine, Calif.) fitted with the Stealth Micro-spotting printhead from TeleChem (Sunnyvale, Calif.). Contact printing technologies include mechanical devices using solid pins, split pins, tweezers, micro-spotting pins and pin and ring. Contact printing technologies are available commercially from a number of vendors including BioRobotics (Boston, Mass.), Genetix (Christchurch, United Kingdom), Incyte (Palo Alto, Calif.), Genetic MicroSystems (Santa Clara, Calif.), Affymetrix (Santa Clara, Calif.), Synteni (Fremont, Calif.), Cartesian Technologies (Irvine, Calif.) and others. Non-contact printing technologies include “ink-jetting” type devices such as those that employ piezoelectrics, bubble-jets, micro-solenoid valves, syringe pumps and the like. Commercial vendors of non-contact printing technologies include Packard Instruments (Meriden, Conn.), Agilent (Palo Alto, Calif.), Rosetta (Kirkland, Wash.), Cartesian Technologies (Irvine, Calif.), Protogene (Palo Alto, Calif.) and others. Both contact and non-contact devices can be used on either homemade or commercial devices capable of three-dimensional movement. Motion control devices from Engineering Services Incorporated (Toronto, Canada), Intelligent Automation Systems (Cambridge, Mass.), GeneMachines (San Carlos, Calif.), Cartesian Technologies (Irvine, Calif.), Genetix (Christchurch, United Kingdom), and others would also be suitable for manufacturing microarrays according to the present invention.

The amount of compound required will depend on the particular nature of the assay, but, for proteins and nucleic acids, attomole amounts usually are sufficient.

The size of the microarray is typically about 1.0-2.0 cm² but may vary over a large range. The array pattern is not critical and can be optimized for a particular device or assay. A typical spot diameter is about 100 um (usually in the range 50-100 um, depending on the method of spotting), with spots placed at a center-to-center spacing of about 140 um (usually in the range 200-1000 um, or separating spots by at least about 10 um), to allow each spot to form at a distinct and separate location on the substrate. In one embodiment, compounds are spotted as microarrays with a column pitch of about of 563 μm and row pitch of about 281 μm. However, in some embodiments very small spots of reagents are deposited; usually spots of less than 10 nl are deposited, in other instances less than 5 nl, 2 nl or 1 nl, and in still other instances, less than 0.5 nl, 0.25 nl, or 0.1 nl. The final spot of dried reagent may be as small as 7 microns in diameter.

In making an array, a solution containing reagents is usually deposited, and the solvent allowed to evaporate leaving a desiccated reagent. The desiccated reagent spots are thus introduced into the device and may be re-solvated by introducing liquid through the flow channel network. A carrier may be introduced to facilitate re-solvation of the reagents, for example they may be co-spotted with a 1-2% BSA solution. BSA may be added to the solution before spotting or BSA can be co-spotted (e.g., under a spot of reagent). The co-deposited BSA also aids in the visualization of the spots useful for the manual alignment of the array to the microfluidic chambers.

Since all the spots are ultimately segregated on the microfluidic device by our specific channel geometry and active valves it is possible to make efficient use of more complex arrays. Two approaches—“co-multispotting” and “neighbor spotting”—are especially useful for introducing more than one solution to the same vicinity, creating complex multiplexed arrays on a MITOMI chip.

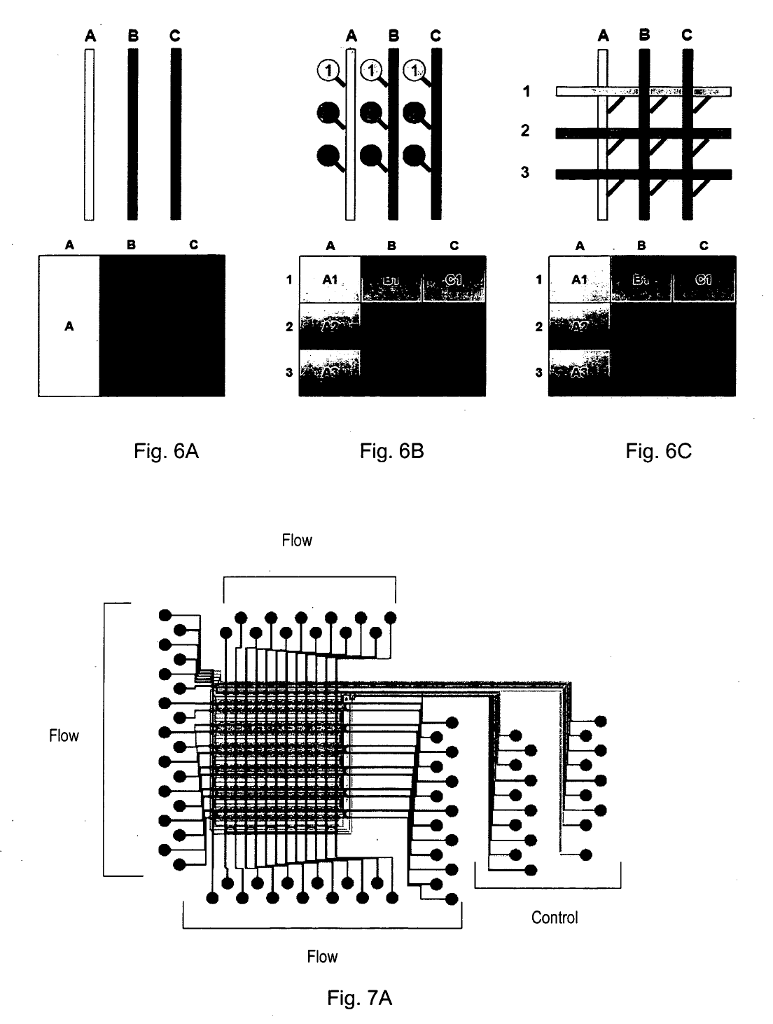

In “co-multispotting” two or more different reagent-containing solutions are deposited on top of one another in sequential rounds of spotting, so that several different components are located in the same place on the array. See, e.g., FIG. 7, Panel B. This figure illustrates cospotting to generate 3-solution combinations of 3 pairs of solutions (Pair 1=A and B; Pair 2=1 and 2 and Pair 3=alpha and beta). If any given spot contains only one member of a pair, the total number of possible combinations is 2³=8. In one embodiment the array is generated by spotting members of the first pair in columns, followed by a second round of spotting of the same or different solutions across rows. In this example, the first two rounds represent a standard two dimensional array of dimensions m×n where m is the number of columns and n the number of rows of the array. Printing of a three dimensional array of shape m×n×o can be accomplished by spotting o copies of the two-dimensional array m×n. So in the case shown in Panel B of FIG. 7, a three-dimensional array of shape m=n=o=2 is spotted on a two dimensional substrate. Likewise any array of higher dimensionality can be printed using the same technique. The deposits of solution A and B spotted in the same round may be spotted in sequence without the need of a wash step between duplicate spots. For any subsequent round of spotting it is preferred, if pins are used for deposit, to wash between every deposited spot due to possible contamination of the pin from the previously deposited spot. Co-multispotting is extremely space efficient since it requires the same area as a standard array, and spots may be spaced with a minimal pitch merely dictated by the pin, spotting robot and fluidic layout to which the array is being aligned. Co-multispotting can also be used to increase reagent concentration per spot by multispotting the same solution several times on the same spot, each time delivering more reagent to the amount already present on the slide.

A second approach to multiplexing by spot deposition is the method of “neighbor-multispotting” depicted in Panel C of FIG. 7. Here instead of spotting the various solutions directly on top of one another, they are spotted immediately adjacent to one another. The total footprint of the neighboring spots is designed to fit into a single reagent chamber of the MITOMI device, and each group of spots is ultimately segregated on the device. Upon resolvation the spotted reagents are allowed to mix by passive diffusion. This approach has the disadvantage of requiring a larger footprint per spot then the co-multispotting method. However, in some applications this disadvantage is outweighed by the elimination of cross-contamination between spotting since a pin, for example, does not touch a previously deposited spot. Eliminating cross-contamination in this fashion allows for significant time savings by reducing the number of wash steps required. Like co-multispotting, neighbor multispotting can be used to increase reagent concentration by depositing neighboring spots containing the same reagent.

In one aspect the invention provides a method of fabricating a microfluidic device by i) positioning an elastomeric block comprising a plurality of chamber recesses and a solid support comprising a microarray of discrete reagent-containing regions so as to align each reagent-containing region with a recess; ii) adhering the block to the solid support so as to produce a plurality of chambers containing reagents. As used herein, the term “elastomeric block” refers to the elastomeric portion of a microfluidic device made using multilayer soft lithography techniques, which has not yet been adhered to a solid support (or substrate. The elastomeric block contains a plurality of “chamber recesses” that, upon attachment of the solid support form chambers in which the solid substrate forms one surface (e.g., the “floor”). A device made from a nonelastomeric material can be aligned with a substrate in essentially the same manner. In one embodiment the microarray has 10 to 5,000 reagent-containing regions, more often 100 to 2400 reagent-containing regions. In one embodiment each reagent containing region contains two or more different reagents. In one embodiment each reagent containing region contains 1, 2 or 3 discrete subregions, each containing a different reagent.

One aspect the invention provides a method of fabricating a microfluidic device by i) depositing reagents on a solid support to produce a microarray of discrete reagent-containing regions; ii) positioning an elastomeric block comprising a plurality of chamber recesses and the reagent-containing regions so as to align each reagent-containing region with a recess; iii) adhering the block to the solid support so as to produce a plurality of chambers containing reagents. In one embodiment the reagents are deposited by contact printing. In one embodiment the reagents are deposited by non-contact printing. In one embodiment the reagents are deposited on the solid support robotically. In one embodiment the microarray has a density of about 100 or more discrete regions per cm². In one embodiment the microarray has a density of about 1000 or more discrete regions per cm².

In one aspect the invention provides a microfluidic device with at least 100 unit cells, each unit cell having a first microfluidic chamber having a substrate, and a reagent in dry form disposed on a reagent-containing region of the substrate where at least 100 unit cells of the device each contains a different reagent, different amounts of a reagent, or a different combination of reagents. It will be recognized that, in some embodiments, &*& the microarray comprises 10 to 1000 different reagents.

### Part 2

### PCR Based Approach for Generating a Linear Expression Vector

In PART 2 we present a purely PCR based approach for generating a linear expression vector, which is highly modular and can easily be scaled up to thousands of target genes. Our approach only requires an ORF as starting material, which may be obtained from a variety of sources including yeast and bacterial genomic DNA or eukaryotic cDNA clones. All other components of the system are commercially procurable oligomers of lengths of up to 100-130 basepairs. Using our two step PCR method large libraries of linear expression ready templates can be synthesized in under a day.

These templates can be used for microarray spotting or flow deposition as described above, or for any process that can be carried out using expression templates. It is further possible to port this approach to on-chip synthesis, by introducing primer pairs and their respective template by co-spotting as mentioned above and running the PCR reaction in situ on-chip.

This method can be used for rapid in situ synthesis of protein using in vitro transcription/translation. This allows us to generate large libraries of proteins to be tested (e.g., for binding activity).

The PCR method adds all necessary 5′ and 3′ UTRs required for the efficient transcription and translation of the template. It is also capable of adding additional amino acids to the 5′ or 3′ terminus of the expressed protein, and extremely useful for generating epitope tagged protein variants.

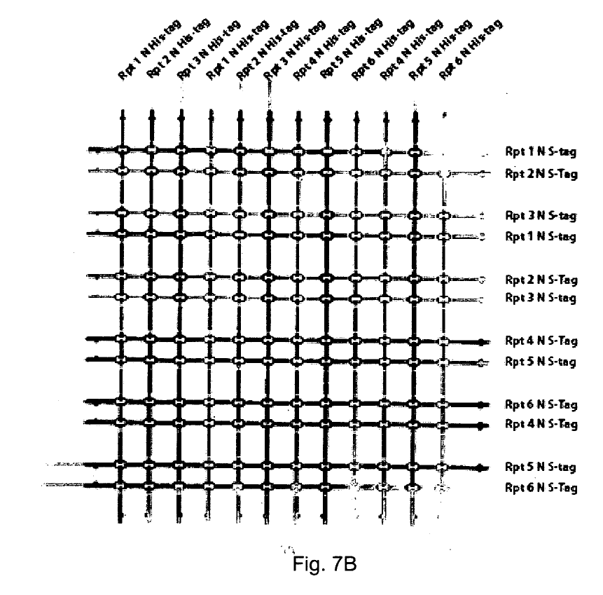

The PCR approach can be carried out using single stranded standard oligomers commercially available from a variety of vendors (Operon, IDT, Eurogentech). Three sets of oligos are used in two sequential PCR steps, in order to add all the required additional sequences for expression (FIG. 8).

Methods for amplification using PCR are well known in the art. Guidance is available in Innis et al., 1989, PCR Protocols: A Guide to Methods and Applications (Academic Press); Ausubel et al., Current Protocols In Molecular Biology, Greene Publishing and Wiley-Interscience, New York (as supplemented through 2006); Sambrook et al. 1989, Molecular Cloning: A Laboratory Manual, 2nd Edition, (Cold Spring Harbor Laboratory Press).

Step 1

The original ORF is extended by overhang extension in each step of the PCR. In the first step the ORF is amplified with gene specific primers (see FIG. 8). The gene specific primers include gene specific primer sequences (i.e., sequence complementary to a strand of the target gene), and a region containing a portion of the 5′ and 3′ UTRs needed for ITT, as well as sites for priming with the second set of primers. Necessary 5′ and 3′ UTRs will vary depending on the in vitro transcription/translation system used, but include start and stop codons (if not included in the amplified ORF sequence) and a Kozak sequence (ribosome binding sequence). The gene specific primers optionally may also carry any expressed sequences tags to be added to the ORF (i.e., so that a fusion protein is encoded). Exemplary tags include a 5× Histidine tag, a 6× Histidine tag and a T7 tag. For example, we have generated a variety of epitope tag variants showing that the addition of sequences to the N and C terminus of the ORF is possible.

Once the first PCR is complete the product may be purified or directly used as template in the second PCR step. Since only a fraction of the first PCR step is needed to seed the second step, dozens of second step PCRs may be run on the template obtained in the first step.

Step 2

The second PCR step consists of two sub-steps taking place in a single tube.

In the first sub-step (Step 2a) the second set of primers (the 5′ and 3′ extension primers) are used to amplify and add additional necessary UTR sequences. The 5′ extension primer includes a region complementary to the corresponding gene-specific primer, a promoter sequence (e.g., T7 promoter) and optionally a beta-globin sequence. Preferably, the a region complementary to the corresponding gene-specific primer encodes a functional sequence. For example, in FIG. 8, the primer regions (PR) adjacent to the Kozak and beta-globin regions preferably comprise a portion of the Kozak and/or beta-globin sequences (i.e., so that there are not intervening elements between the globin and Kozak sequences that might disrupt the spacing of the elements. The 3′ extension primer includes a 3′ sequence complementary to one strand of gene-specific primer, a poly(A) sequence and a terminator sequence (e.g., T7 terminator). The primer regions (PR) may comprise a portion of the stop codons, tag sequences, and/or poly A site to minimize the length of the primers and maintain optimal spacing between UTR elements. The second set of primers also includes sites for priming with a third set of primers. The extension primers are typically 80 tol 130 bases in length.

This step can be accomplished with a low concentration of primers and, for example, 10 amplification cycles. Upon completion of the first sub-step, the second sub-step (Step 2b) is carried out.

The PCR reaction is spiked with the final amplification primers, 5′amp and 3′amp and run for an additional 30 cycles. These primers amplify only the full length templates. Additionally the primers may be coupled to moieties such as biotin (allowing the product to be bound by streptavidin) or a variety of fluorophores (allowing the protein to be visualized).

Due to the use of readily available primers the method is highly modular and has been applied to both eukaryotic based and prokaryotic based in vitro protein synthesis by re-designing the 5′extension primer introducing the required sequence components.

In one embodiment, conditions (temperature, time, etc.) are the same for each PCR steps remain the same and do not need to be re-optimized.

This approach does not require extensive purification steps or difficult to prepare double stranded extension DNA pieces. There are also no problems with primer dimer formation due to the separation of the two PCR steps and the sub segregation of the second step. This makes it easy to use and avoids time consuming and labor intensive purification steps such as gel purification or alcohol precipitation. The promoter structure may be changed to work with most phage RNA polymerases such as T7, T3 and Sp6. Additional yield enhancing structures such as a beta-globin and poly (A) tails may be added to the 5′ and 3′ UTR respectively.

It is also possible to perform this reaction as a single step PCR reaction without the need of spiking reagents. This is possible since the annealing temperatures have been staggered so that the various steps can be performed in the same tube sequentially by gradually reducing the annealing temperature.

Additionally, the PCR method is capable of adding expressed sequences to the N or C-terminus of the expressed protein and may be used to generate chimeras and other changes in the protein primary structure.

### EXAMPLE

Linear expression templates were generated by a two step PCR method (FIG. S2) in which the first step amplifies the target sequence and the second step adds required 5′UTR and 3′UTR for efficient ITT. Pho4 N or C-His tagged and Cbf1 N or C-His tagged versions were amplified 1 from yeast genomic DNA as follows: The first step PCR reaction contained 1 μM of each gene specific primer, 10 ng μL-1 yeast genomic DNA (SeeGene), 200 μM of each dNTP and 2.5 units of TAQ enzyme mixture (Expand High Fidelity PCR system, Roche) in a final volume of 50 μL. The reaction was cycled for 4 min at 94° C., followed by 30 cycles of 30 s at 94° C., 60 s at 53° C. and 90 s at 72° C. followed by a final extension of 7 min at 72° C. The products were then purified on spin columns (QIAquickPCR, Qiagen) and eluted in 75 μL of 10 mM TrisCl, pH 8.5. The purified product then served as template in the second PCR reaction using 2 μL first PCR product, 5 nM 5′ext1 primer, 5 nM 3′ext2 primer, 200 μM of each dNTP and 2.5 units of TAQ enzyme mixture (Expand High Fidelity PCR system, Roche) in a final volume of 100 μL. The reaction was cycled for 4 min at 94° C. followed by 10 cycles of 30 s at 94° C., 60 s at 53° C. and 90 s at 72° C. followed by a final extension of 72° C. for 7 min. After this first round of extension 2 μL of 5 μM 5′finalCy5 and 5 μM 3′final in dH2O were added to each reaction and cycling was continued immediately at 94° C. for 4 min followed by 30 cycles of 30 sec at 94° C., 60 sec at 50° C. and 90 s at 72° C. followed by a final extension of 72° C. for 7 min. The final product was then purified on spin columns and eluted in 100 μL 10 mM TrisCl, pH8.5 or used directly in ITT reactions. Linear expression templates for MAX iso A, MAX iso B were synthesized essentially as above except that bacterial cDNA clones (MGC) lysed in 2.5 μL Lyse n′ Go buffer (Pierce) at 95° C. for 7 min where used as template in an Expand High Fidelity PCR reaction (Roche). The first PCR product was purified using the Qiaquick 96 PCR purification kit (Qiagen) and eluted in 80 μL of 10 mM TrisCl, pH 8.5. To assess the fidelity of these multi-step PCR reactions and to ascertain that no point mutations accumulated during the reaction we submitted final products of MAX iso B notag, MAX iso B C-His, PHO4 C-His and CBF1 N-His to sequencing (Biotech Core). The resulting sequences showed extremely high-fidelity with no accumulation of point mutations (data not shown).

### Part 3

### Combinatorial Protein Expression and Protein Production

In PART 3 we present a method for the flow dependent surface deposition of linear expression templates which are transcribed and translated for the in situ generation of protein. In one aspect, multiple proteins are expressed in the same compartment and protein-protein interactions are analyzed. The method also may be used for efficient generation of a protein or protein(s).

Hundreds or thousands of unique assays per microfluidic device can be carried out based on the deposition of molecules on the surface of the microfluidic channels or chambers in the device. In one approach linear expression templates, such as the dsDNA PCR products described in Part 2, supra, are used as templates from which protein is synthesized by in vitro transcription/translation (ITT). When multiple proteins are expressed in the same compartment interactions between them can be studied.

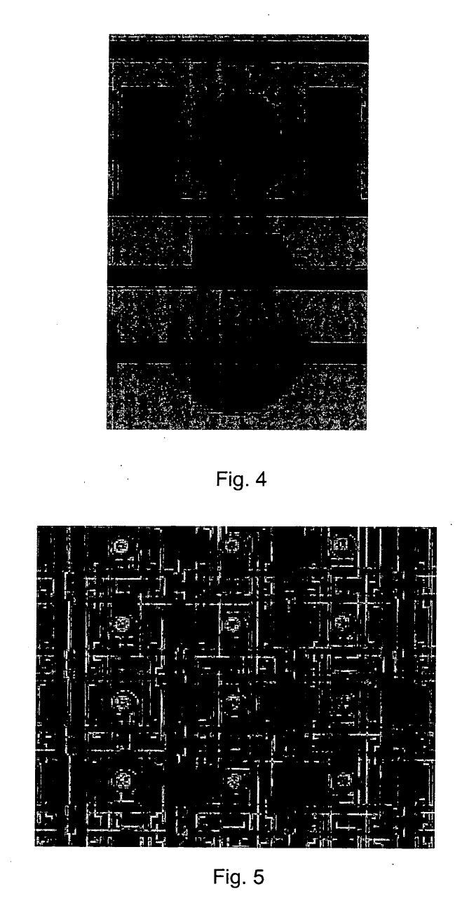

FIG. 6 is a schematic and illustrates ways in which various templates may be deposited in the device. Linear templates are bound to the substrate. Binding of the template can be accomplished in a number of ways. A convenient method for attachment is via the interaction of a ligand (tag) covalently bound to the DNA template and a corresponding anti-ligand on the substrate. For example, nucleic acid templates tagged by a 3′ biotin moiety may be deposited on a streptavidin coated surface. Methods for preparing biotinylated primers are well known and reagents are available commercially, but any number of ligands can be attached to an expression template (e.g., using a tagged nucleic acid primer for amplification) and be used to immobilize the template to a surface to which a corresponding anti-ligand is attached. In another example, nucleic acid templates tagged by a sugar may be deposited on a lectin coated surface. Alternatively, a portion of the template can be made single stranded (e.g., by restriction or by ligation of a partially single stranded nucleic acid to the template) and the template immobilized by hybridization to a complementary binding sequence immobilized on the substrate. Templates can also be bound non-specifically to a substrate derivatized with epoxy, aldehyde or amine using methods known in the art.

FIG. 6A illustrates a simple pattern generated by introducing templates coding for different templates into parallel columns on the device. Linear templates are being bound by surface bound streptavidin anchoring them in place. In this illustration a different template (A, B, C) is deposited in each of three columns.

FIG. 6C shows introduction of another set of templates (1, 2, 3) in channels that intersect the columns. Usually when reagents A, B, C are flowed through the column channels, valves are closed to isolate the columns from row channels and, conversely, when reagents 1, 2, 3 are flowed through the row channels, valves are closed to isolate the rows from column channels. It is possible, however, to flow the second solution (e.g., through rows) without isolating the columns, since the substrate of the columns will be fully or sufficiently saturated with reagents A-C. Thus, combinatorics is achieved by introducing another set of templates perpendicular to the initial set of templates. This creates a complete matrix of all possible combinations of the two sets of templates introduced so that protein synthesized from the deposited templates may be tested for binary interactions (2.2.2) by simple diffusion. Individual combinations are compartmentalized from one another using microfluidic valves.

The flow channels will be in fluidic communication with sources for each reagent (e.g., A, B, C, 1, 2, 3). The sources can be reservoirs integral to the device, or reservoirs external to the device connected to flow channels via an inlet. The device will therefore comprise a plurality of reservoirs and/or inlets sufficient to provide reagents.

It is contemplated that in some embodiments, more than two flow channels may cross at an intersection allowing higher level interactions to be tested.

FIG. 6B illustrates use of a combination of the methods of microarray based programming described supra in Part 1 and flow deposition on a single device. For example, an array of templates (1, 2, 3) can be spotted on a substrate as described in Part 1, and aligned with an elastomeric block so that the templates are contained in a chamber (a “reagent chamber”) that can be isolated from a flow channel (or intersection of flow channels) in which other templates are flowed and immobilized.

Once the template (or templates) is immobilized on the substrate, transcription and translation may be carried out by introducing wheat germ extract (or other cell-free transcription/translation system). Typically the device is incubated at 30° C. for 90 min to complete protein synthesis. It will be understood that individual combinations of templates are compartmentalized from one another using microfluidic valves. Valves separating a reagent chamber (containing a template or other spotted reagent) can be opened to allow the ITT system to contact the spotted template. In some embodiments the reagent chamber contains an agent other than a template. For example, before, during or following synthesis of protein(s) from immobilized templates, the agent in the reagent chamber can be re-solvated and the effect of the agent in protein activity or protein interactions can be assessed.

In one approach, a library of agents can be spotted and the effect of each agent on the same combination of proteins can be assessed. In another approach, each reagent chamber can have the same agent and the effect of a single agent on numerous different combinations of proteins can be assessed.

The fluidic layout may be designed to accommodate any possible combination of methods and complexity. Using flow deposition of linear expression templates allows for the facile generation of hundreds of combinatoric protein assays. It provides a second efficient way to introduce information into a microfluidic device. Two sets of samples may be easily tested in all possible combinations against one another. Flow deposition therefore is the most rapid and simplest ways of generating complex combinatoric binary assays since it does not require additional bench-top equipment such as a micro-array spotter and is performed on the same platform as all remaining downstream assays.

Thus, the invention provides a microfluidic device having (a) a first plurality of microfluidic flow channels each channel comprising a substrate; (b) a second plurality of microfluidic flow channels, each channel comprising a substrate, the second flow channels intersecting the first flow channels to define an array of reaction sites; each of the channels having expression templates encoding proteins are immobilized on their surfaces. At least one channel in the first plurality comprises an immobilized expression template that differs from the expression template immobilized in at least one channel in the second plurality. In practice, usually many of the expression templates immobilized in the two sets of channels will differ from each other, in order to maximize the number of different protein combinations to be expressed. Usually some of the channels in each set will be duplicates of other channels, negative controls, etc., and in some cases the set of expression templates in the two sets of channels may overlap. The device will also include sets of microfluidic valves that can isolate reaction sites from each other so that pairwise interactions can be analyzed. Generally, the area isolated by these isolation valves encompasses more than the area of intersection of channels. That is, usually at least some additional portion of one of the channels is included in the isolated area. The area at which the channels intersect will usually contain only, or primarily, one of the two expression templates. This is because the first template flowed across the intersection may saturate or largely saturate the sites on the substrate to which the first expression template binds, blocking binding by the second expression template. Thus, each set of valves isolates a reaction region comprising a defined combination of expression templates (i.e., an expression template that is immobilized in a channel from the first plurality of channels and a different expression template that is immobilized in a channel from the first plurality of channels). In various embodiments the isolated reaction regions include at least 50 different defined combinations of expression templates and therefore at least 50 different combinations of proteins. In some embodiments the number is at least 100, at least 200 or at least 500.

The number of unique expression templates in the first plurality of microfluidic flow channels is at least one, but is usually at least 5, more usually at least 10, more usually at least 20, more usually at least 50, and sometimes at least 100 or more. The number of unique expression templates in the second plurality of microfluidic flow channels is at least one, but is usually at least 5, more usually at least 10, more usually at least 20, more usually at least 50, and sometimes at least 100 or more. The number of unique expression templates in first plurality and second plurality of microfluidic flow channels taken together is usually at least 10, more usually at least 20, more usually at least 50, and sometimes at least 100 or more.

It will be appreciated that the device is useful for analyzing protein-protein interactions by introducing a cell-free transcription translation system into the regions comprising defined combinations of expression templates, actuating valves to isolate reaction regions, and maintaining the device under conditions in which protein synthesis occurs and thereby producing proteins encoded by the expression templates; and detecting the interaction between said proteins.

Detection of proteins is accomplished using any number of art known methods. In one approach, one or both proteins is labeled (e.g., with a fluorescent dye or macromolecule). This can be achieved by either building a chimeric protein making use of the various GFP variants or other fluorescent proteins. A second approach includes site or residue specific incorporation of a modified amino acid via a modified tRNA (e.g., charged with an amino acid linked to a dye). Alternatively, an amino reactive fluorescent dye or quantum dot can be included in the reaction mixture to label a protein(s).

In another approach optical methods for detecting protein-protein interactions are used. Examples include FRET based measurements (where a signal is generated by bringing two proteins in close proximity to one another—due to interaction—which in turn allows the dyes attached to the molecules to function as a FRET donor and acceptor pair); fluorescent correlation spectroscopy (FCS) (based on measuring the diffusional coefficient of a protein by interrogation how fast a molecule diffuses through a focused excitation spot); fluorescence cross-correlation spectroscopy (FCCS); and surface plasmon resonance which detects interactions on a surface as a function of refractive index changes of that surface. Other standard methods such as ELISA etc. may also be used to detect interacting species.

Although described above primarily in relation to protein-protein interactions, it is contemplated that the flow deposition methods of the invention can be applied to any set of pairwise combinations of molecules. In some embodiments one molecule is an expression template. In some embodiments none of the molecules is an expression template. In some embodiments pairwise combinations of an expression template and none of the molecules is an expression template.

Protein Production

In a related method, surface linked linear expression templates are used for the continuous synthesis of protein, a method otherwise not achievable bench-top. Using continuous flow-synthesis of protein constantly introduces new ITT mixture on top of the deposited DNA, keeping the synthesis rate at a near optimum. Thus, the immobilized expression template is used as a template for continuous, rather than batch reactions, by using a constant flow of ITT mixture over the deposited DNA. The protein produced may be concentrated and purified either off-chip or on-chip depending on the downstream requirements. Protein production can proceed for extended periods of time, merely limited by the stability of the deposited DNA templates. It is also possible to de-couple the transcription and translation reactions spatially on the device, increasing the efficiency of each individual step and thus allowing for higher total synthesis yields.

Any composition containing reagents sufficient for cell-free transcription and translation (ITT composition) can be used. Preferably the ITT composition is a Wheat Germ extract. ITT compositions and methods for their use for protein synthesis are well known in the art. “Conditions” in which protein synthesis and transcription occur refers to a temperature, pH, etc. appropriate for the ITT composition.

With flow deposition the introduced templates are automatically affinity purified, eliminating the need for any off-chip upstream purification. It also concentrates the sample on-chip due to the high surface to volume ratio intrinsic to microfluidic devices.

Sample concentration has the advantage that protein synthesis is more efficient using deposited template since protein yield is strongly correlated with DNA input concentration. Therefore on-chip synthesis performs better than off-chip synthesis. Continuous flow synthesis is, in principal, limited only by the linear expression template surface retention and linear template resilience to DNAse dependent degradation. As noted previously, the fluidic layout can be designed to decouple the transcription from the translation step, ultimately increasing protein yield to considerable amounts.

In designing a system in which flow deposited DNA is transcribed, the effect of possible steric hindrance (from close packing of the template to the surface) must be considered, since the entire DNA strand must be accessible for transcription. It is thus desirable to generate a surface chemistry that allows access to the DNA by RNAP over the entire length of the DNA strand to be transcribed. In one approach this is achieved by the use of streptavidin as the surface linker and spacer. The high surface to volume ratio present in microfluidic devices allows protein synthesis to proceed efficiently from flow deposited DNA is a high surface to volume ratio only present in microfuidic devices. The high ratio is required in order to achieve the necessary DNA concentration. For the flow deposition scheme we successfully synthesized protein both in a single batch and multiple batch reaction, showing that continuous protein synthesis is feasible.

Variations include the deposition and assaying of molecules other than linear expression templates as well as varying the surface chemistries to achieve different types of patterning. Furthermore the fluidic geometry may be adjusted to requirements of the assay to be run.

### Part 4

### General Materials and Fabrication Methods

The methods used in fabrication of a microfluidic device will vary with the materials used, and include soft lithography methods, microassembly, bulk micromachining methods, surface micro-machining methods, standard lithographic methods, wet etching, reactive ion etching, plasma etching, stereolithography and laser chemical three-dimensional writing methods, modular assembly methods, replica molding methods, injection molding methods, hot molding methods, laser ablation methods, combinations of methods, and other methods known in the art or developed in the future. A variety of exemplary fabrication methods are described in Fiorini and Chiu, 2005, “Disposable microfluidic devices: fabrication, function, and application” Biotechniques 38:429-46; Beebe et al., 2000, “Microfluidic tectonics: a comprehensive construction platform for microfluidic systems.” Proc. Natl. Acad. Sci. USA 97:13488-13493; Rossier et al., 2002, “Plasma etched polymer microelectrochemical systems” Lab Chip 2:145-150; Becker et al., 2002, “Polymer microfluidic devices” Talanta 56:267-287; Becker et al., 2000, “Polymer microfabrication methods for microfluidic analytical applications” Electrophoresis 21:12-26; U.S. Pat. No. 6,767,706 B2, e.g., Section 6.8 “Microfabrication of a Silicon Device”; Terry et al., 1979, A Gas Chromatography Air Analyzer Fabricated on a Silicon Wafer, IEEE Trans, on Electron Devices, v. ED-26, pp. 1880-1886; Berg et al., 1994, Micro Total Analysis Systems, New York, Kluwer; Webster et al., 1996, Monolithic Capillary Gel Electrophoresis Stage with On-Chip Detector in International Conference On Micro Electromechanical Systems, MEMS 96, pp. 491496; and Mastrangelo et al., 1989, Vacuum-Sealed Silicon Micromachined Incandescent Light Source, in Intl. Electron Devices Meeting, IDEM 89, pp. 503-506.

In preferred embodiments, the device is fabricated using elastomeric materials. Fabrication methods using elastomeric materials will only be briefly described here, because elastomeric materials, methods of fabrication of devices made using such materials, and methods for design of devices and their components have been described in detail (see, e.g., Thorsen et al., 2001, “Dynamic pattern formation in a vesicle-generating microfluidic device” Phys Rev Lett 86:4163-6; Unger et al., 2000, “Monolithic microfabricated valves and pumps by multilayer soft lithography” Science 288:113-16; Linger et al., 2000, Science 288:113-16; U.S. Pat. No. 6,960,437 (Nucleic acid amplification utilizing microfluidic devices); U.S. Pat. No. 6,899,137 (Microfabricated elastomeric valve and pump systems); U.S. Pat. No. 6,767,706 (Integrated active flux microfluidic devices and methods); U.S. Pat. No. 6,752,922 (Microfluidic chromatography); U.S. Pat. No. 6,408,878 (Microfabricated elastomeric valve and pump systems); U.S. Pat. No. 6,645,432 (Microfluidic systems including three-dimensionally arrayed channel networks); U.S. Patent Application publication Nos. 2004/0115838, 20050072946; 20050000900; 20020127736; 20020109114; 20040115838; 20030138829; 20020164816; 20020127736; and 20020109114; PCT patent publications WO 2005/084191; WO05030822A2; and WO 01/01025; Quake & Scherer, 2000, “From micro to nanofabrication with soft materials” Science 290: 1536-40; Xia et al., 1998, “Soft lithography” Angewandte Chemie-International Edition 37:551-575; Unger et al., 2000, “Monolithic microfabricated valves and pumps by multilayer soft lithography” Science 288:113-116; Thorsen et al., 2002, “Microfluidic large-scale integration” Science 298:580-584; Chou et al., 2000, “Microfabricated Rotary Pump” Biomedical Microdevices 3:323-330; Liu et al., 2003, “Solving the “world-to-chip” interface problem with a microfluidic matrix” Analytical Chemistry 75, 4718-23,” Hong et al, 2004, “A nanoliter-scale nucleic acid processor with parallel architecture” Nature Biotechnology 22:435-39; Fiorini and Chiu, 2005, “Disposable microfluidic devices: fabrication, function, and application” Biotechniques 38:429-46; Beebe et al., 2000, “Microfluidic tectonics: a comprehensive construction platform for microfluidic systems.” Proc. Natl. Acad. Sd. USA 97:13488-13493; Rolland et al., 2004, “Solvent-resistant photocurable “liquid Teflon” for microfluidic device fabrication” J. Amer. Chem. Soc. 126:2322-2323; Rossier et al., 2002, “Plasma etched polymer microelectrochemical systems” Lab Chip 2:145-150; Becker et al., 2002, “Polymer microfluidic devices” Talanta 56:267-287; Becker et al., 2000, “Polymer microfabrication methods for microfluidic analytical applications” Electrophoresis 21:12-26; Terry et al., 1979, A Gas Chromatography Air Analyzer Fabricated on a Silicon Wafer, IEEE Trans, on Electron Devices, v. ED-26, pp. 1880-1886; Berg et al., 1994, Micro Total Analysis Systems, New York, Kluwer; Webster et al., 1996, Monolithic Capillary Gel Electrophoresis Stage with On-Chip Detector in International Conference On Micro Electromechanical Systems, MEMS 96, pp. 491496; and Mastrangelo et al., 1989, Vacuum-Sealed Silicon Micromachined Incandescent Light Source, in Intl. Electron Devices Meeting, IDEM 89, pp. 503-506; and other references cited herein and found in the scientific and patent literature.

Methods of fabrication of complex microfluidic circuits using elastomeric are known and are described in Unger et al., 2000, Science 288:113-116; Quake & Scherer, 2000, “From micro to nanofabrication with soft materials” Science 290: 1536-40; Xia et al., 1998, “Soft lithography” Angewandte Chemie-International Edition 37:551-575; Unger et al., 2000, “Monolithic microfabricated valves and pumps by multilayer soft lithography” Science 288:113-116; Thorsen et al., 2002, “Microfluidic large-scale integration” Science 298:580-584; Chou et al., 2000, “Microfabricated Rotary Pump” Biomedical Microdevices 3:323-330; Liu et al., 2003, “Solving the “world-to-chip” interface problem with a microfluidic matrix” Analytical Chemistry 75, 4718-23,“and other references cited herein and known in the art.

All publications and patent documents (patents, published patent applications, and unpublished patent applications) cited herein are incorporated herein by reference as if each such publication or document was specifically and individually indicated to be incorporated herein by reference. Citation of publications and patent documents is not intended as an admission that any such document is pertinent prior art, nor does it constitute any admission as to the contents or date of the same. The invention having now been described by way of written description and example, those of skill in the art will recognize that the invention can be practiced in a variety of embodiments and that the foregoing description and examples are for purposes of illustration and not limitation of the following claims.

We claim:

1. A method of fabricating a microfluidic device comprising
i) positioning (a) an elastomeric block comprising a plurality of chamber recesses and (b) a solid support comprising a microarray of discrete reagent-containing regions, so as to align each reagent-containing region with a chamber recess,
ii) adhering the block to the upper surface of the solid support so as to produce a plurality of chambers, wherein in each chamber the upper surface of the solid support provides one surface of the chamber and the inner surfaces of a chamber recess provides other surfaces of the chamber;
wherein each reagent-containing region contains two or more discrete subregions, and
wherein at least two subregions in each reagent-containing region contain different reagents.

2. The method of claim 1 wherein the solid support is epoxy-functionalized glass.

3. The method of claim 1 wherein each discrete reagent-containing region contains three discrete subregions, each of which contains a different reagent.

4. The method of claim 1 wherein each discrete reagent-containing region contains four or more discrete subregions.

5. The method of claim 1 wherein the reagents are deposited by contact printing.

6. The method of claim 1 wherein the microarray has a density of 100 or more discrete regions per cm².

7. The method of claim 6 wherein the microarray has a density of 1000 or more discrete regions per cm².

8. The method of claim 1 wherein the microarray includes 10 to 500 different reagents.

9. The method of claim 1 wherein the reagents are proteins and/or nucleic acids.

10. A microfluidic device comprising a plurality of isolated reaction sites wherein one surface of the reaction site is formed by a solid support and each isolated reaction site comprises a reagent-containing region on said surface
wherein the reagent-containing regions contain two or more discrete subregions, and
wherein at least two subregions in each reagent containing region contain different reagents.

11. The device of claim 10 that comprises a plurality of reaction chambers wherein one surface of chamber is formed by a solid support and said surface comprises a reagent-containing region
wherein the reagent-containing region contains two or more discrete subregions, and
wherein at least two subregions in each reagent containing region contain different reagents.

12. The device of claim 10 wherein the solid support is epoxy-functionalized glass.

13. A microfluidic device comprising a plurality of isolated reaction sites wherein one surface of the reaction site is formed by a solid support and each isolated reaction site comprises a reagent-containing region on said surface
wherein the reagent-containing regions contain a first reagent deposited on the solid support and a second reagent deposited on top of the first reagent.

14. The device of claim 13 wherein the first and second reagents are different.

15. The device of claim 14 wherein reaction sites on the array comprise a dilution series of one reagent.

16. A microfluidic device, comprising
(a) a first plurality of microfluidic flow channels each channel comprising a substrate;
(b) a second plurality of microfluidic flow channels, each channel comprising a substrate, the second flow channels intersecting the first flow channels to define an array of reaction sites;
wherein expression templates encoding proteins are immobilized on said substrates; and
wherein at least one channel in the first plurality comprises an immobilized expression template that differs from the expression template immobilized in at least one channel in the second plurality; and
(c) sets of isolation valves selectively actuatable to fluidically isolate reaction sites from each other, wherein said sets of valves each isolate a reaction region comprising a defined combination of expression templates, wherein the defined combinations each comprise an expression template that is immobilized in a channel from the first plurality and a different expression template that is immobilized in a channel from the first plurality.

17. The device of claim 16 wherein in aggregate, said isolated reaction regions comprise at least 50 different defined combinations of expression templates.

18. The device of claim 16 wherein the number of unique expression templates in the first plurality of microfluidic flow channels is at least 10.

19. The device of claim 16 wherein the number of unique expression templates in the second plurality of microfluidic flow channels is at least 10.

20. The device of claim 16 wherein the number of unique expression templates in the first plurality and second plurality of microfluidic flow channels, taken together, is at least 10.

21. The device of claim 16 additionally comprising at least one set of intersecting channels in which both channels in the set comprise the same expression template.

22. A method for analyzing protein-protein interactions comprising
(i) in a device according to claim 16 introducing a cell-free transcription translation system into the regions comprising defined combinations of expression templates, actuating valves to isolate reaction regions, and maintaining the device under conditions in which protein synthesis occurs and thereby producing proteins encoded by the expression templates; and
(ii) detecting the interaction between said proteins.

23. A method for producing a protein comprising,
in a microfluidic device comprising a microfluidic flow channel comprising a substrate on which an expression template encoding a protein is immobilized;
flowing a composition containing reagents sufficient for cell-free transcription and translation (ITT composition) through the channel, under conditions in which transcription of the expression template occurs and the encoded protein is produced; and,
collecting the encoded protein from the flow channel.

24. The method of claim 23 in which the ITT composition is Wheat Germ extract.

25. The method of claim 24 in which the microfluidic device comprises a plurality of flow channels and wherein the expression templates immobilized in at least two of the flow channels is different.
